SEQUENCE LISTING (2 sequences)

SEQ ID NO: 1 - protein, 48 aa, Homo sapiens
GPDICGPGTKKVHVIFNYKGKNVLINKDIRCKDDEFTHLYTLIVRPDN

SEQ ID NO: 2 - DNA, 144 bp, Homo sapiens
ggtcccgacatctgtggccctggcaccaagaaggttcatgtcatcttcaactacaagggcaagaacgtgctgatcaacaaggacatccgttgcaaggatgatgagtttacacacctgtacacactgattgtgcggccagacaac

(19) United States
(12) Patent Application Publication
(10) Pub. No.: US 2013/0296242 A1
(43) Pub. Date: Nov. 7, 2013

(54) MEDICATION AND THERAPY TREATING OF CHOROIDAL NEOVASCULARIZATION

(71) Applicant: NATIONAL SUN YAT-SEN UNIVERSITY, Kaohsiung (TW)

(72) Inventors: Ming-Hong TAI, Kaohsiung (TW); Youn-Shen BEE, Kaohsiung (TW); San-Cher CHEN, Kaohsiung (TW)

(73) Assignee: NATIONAL SUN YAT-SEN UNIVERSITY, Kaohsiung (TW)

(21) Appl. No.: 13/796,809

(22) Filed: Mar. 12, 2013

(30) Foreign Application Priority Data
May 3, 2012 (TW) 101115833

(51) Int. Cl. C07K 14/47

(52) U.S. Cl. CPC C07K 14/4728
USPC 514/13.3

(57) ABSTRACT

A medication for treating choroidal neovascularization comprises an amino acid fragment that derives from an angiogenesis inhibiting factor and comprises amino acid sequences as set forth in SEQ ID NO: 1. Moreover, a therapy of treating choroidal neovascularization by administering the said medication to an individual, with the medication comprising 0.1-1000 μg of the amino acid fragment in 1 ml solvent.

### BACKGROUND OF THE INVENTION

1. Field of the Invention

The present invention relates to a medication for eyes and, more particularly, to a medication for treating choroidal neovascularization.

2. Description of the Related Art

Choroidal neovascularization (CNV) is the primary cause for vision loss in patients with high myopia, diabetic retinopathy, neovascular glaucoma (NVG) and age-related macular degeneration. Generally, treatments for choroidal neovascularization and corneal neovascularization are quite diverse, and which includes laser photocoagulation surgery, photodynamic therapy (PDT), and surgical resection. However, the said treatments for choroidal neovascularization all request invasive procedures, being limited in treating of angiogenesis and easy to lead to complications and side effects, such as wounds and inflammation.

Protein drug is a new trend of targeted therapy. Recently, increasing use of recombinantly expressed therapeutic proteins in the pharmaceutical industry becomes highlighted issues such as their stability during long-term storage, means of safety and efficacious delivery that avoid adverse immunogenic side effects. As an example, protein drugs can be used in therapies of malignancy and rheumatoid arthritis (RA) via their special targeting activity.

Conventional protein drugs for choroidal neovascularization include avastin and lucentis, but they are inconvenient in delivery for most patients since avastin and lucentis have to be applied to eyeball directly through syringes. Furthermore, the conventional protein drugs, avastin and lucentis, all associates with less severe health side effects. Avastin will lead to plenty of side effects, such as nosebleeds, hypertension, vein thrombosis and slight proteinuria, and rarely for gastrointestinal hemorrhage and perforation. Yet, lucentis will cause eye infection, subconjunctival haemorrhage, intraocular hemorrhage and retinal detachment. Hence, the conventional protein drugs have plenty disadvantages in practical uses.

Calreticulin is a protein that is encoded by the CALR gene in humans, suggesting that it acts as a significant modulator of the regulation of gene transcription by nuclear hormone receptors and it has an important role on anti-tumor. Calreticulin is consisted of 417 amino acids, comprising vasostatin (also known as VS180) being a fragment ranged from 1^{st} to 180^{th} of N-terminal amino acids and being capable of inhibiting angiogenesis.

Sheu et al (Inhibition of choroidal neovascularization by topical application of angiogenesis inhibitor vasostatin, Molecular Vision 2009; 15:1897-1905) discloses a kind of eye drops, with recombinantly expressed VS180 (such as E. coli expressed VS180) act as an inhibitor of angiogenesis. However, the recombinantly expressed VS180 to Sheu et al is poor in delivering to cells due to a high molecular weight thereof. Also, the recombinantly expressed VS180 is not easy to bind with vascular endothelial cells, and thus, the efficiency of inhibition on angiogenesis is low.

In addition, the solubility and stability of the VS180 is low in water due to a molecular weight of the VS180, so that sediments are usually occurred as the VS180 is dissolved in water as being manufacturing into eye drops. With such, the binding ability to vascular endothelial cells, as well as inhibiting ability to angiogenesis are all reduced. Accordingly, the recombinantly expressed VS180 requests a tagged protein, such as thioredoxin (TRX), to increase the solubility thereof. However, thioredoxin is derived from a bacteria protein. A thioredoxin combined recombinantly expressed VS180 (TRX-VS180) usually induces immune responses in hosts, including red eyes and itch, when it is delivered to individuals, still being poor in practical use.

Hence, it is need to provide a new medication, which shares preferable binding ability with target cells and preferable inhibiting ability to angiogenesis, and will not lead to negative immune response to individuals.

### SUMMARY OF THE INVENTION

An objective of the present invention is to provide a medication for treating choroidal neovascularization that can precisely boost inhibiting ability of VS180 on neovascularization.

Another objective of the present invention is to provide a medication for treating choroidal neovascularization that can increase binding ability of VS180 with vascular endothelial cell.

A further objective of the present invention is to provide a medication for treating choroidal neovacularization that can improve the solubility of VS180 in water.

The present invention fulfills the above objectives by providing a medication for treating choroidal neovascularization comprising an amino acid fragment that derives from an angiogenesis inhibiting factor and comprises amino acid sequences as set forth in SEQ ID NO: 1.

The said medication for treating choroidal neovascularizatio further comprises a solvent, with the amino acid fragment being dissolved in the solvent, wherein the solvent is 0.85-0.90% saline.

Preferably, 0.1-1000 μg of the amino acid fragment is dissolved in 1 ml of the solvent.

Preferably, the medication is in a type of a drop.

The present invention further provides a therapy of treating choroidal neovascularization by administering said medication for treating choroidal neovascularizatio to an individual in need, with said medication for treating choroidal neovascularizatio comprising 0.1-1000 μg the amino acid fragment in 1 ml solvent.

Preferably, the solvent is 0.85-0.90% saline.

The present invention will become clearer in light of the following detailed description of illustrative embodiments of this invention described in connection with the drawings.

### BRIEF DESCRIPTION OF THE DRAWINGS

The illustrative embodiments may best be described by reference to the accompanying drawings where:

FIG. 1 is a diagram illustrating amino acid fragments of VS180, VS48 and VS132;

FIG. 2 is a bar chart illustrating immune responses in groups B1 and B2;

FIG. 3 shows photos of groups C1 and C2 illustrating inhibiting efficiencies of choroidal neovascularization therein;

FIG. 4 is bar chart illustrating inhibiting efficiencies of choroidal neovascularization in the groups C1 and C2;

FIG. 5 shows photos of groups D1 and D2 illustrating inhibiting efficiencies of choroidal neovascularization therein;

FIG. 6 is a bar chart illustrating binding rate of groups E1 and E2 with HUVEC;

FIG. 7 is a bar chart illustrating binding rate of groups E1 and E2 with NIH3T3;

FIG. 8 shows photos of groups F1 to F3 illustrating vascular endothelial cell migration thereof;

FIG. 9 is a bar chart illustrating vascular endothelial cell migration in groups F1 to F3;

FIG. 10 shows photos of groups F4 to F6 illustrating vascular endothelial cell columns therein;

FIG. 11 is a bar chart illustrating vascular endothelial cell columns in groups F4 to F6;

FIG. 12 shows photo of groups G1 to G3 illustrating angiogenosis;

FIG. 13 is a bar chart illustrating growing rates in groups G1 to G3.

All figures are drawn for ease of explanation of the basic teachings of the present invention only; the extensions of the figures with respect to number, position, relationship, and dimensions of the parts to form the preferred embodiments will be explained or will be within the skill of the art after the following teachings of the present invention have been read and understood. Further, the exact dimensions and dimensional proportions to conform to specific force, weight, strength, and similar requirements will likewise be within the skill of the art after the following teachings of the present invention have been read and understood.

### DETAILED DESCRIPTION OF THE INVENTION

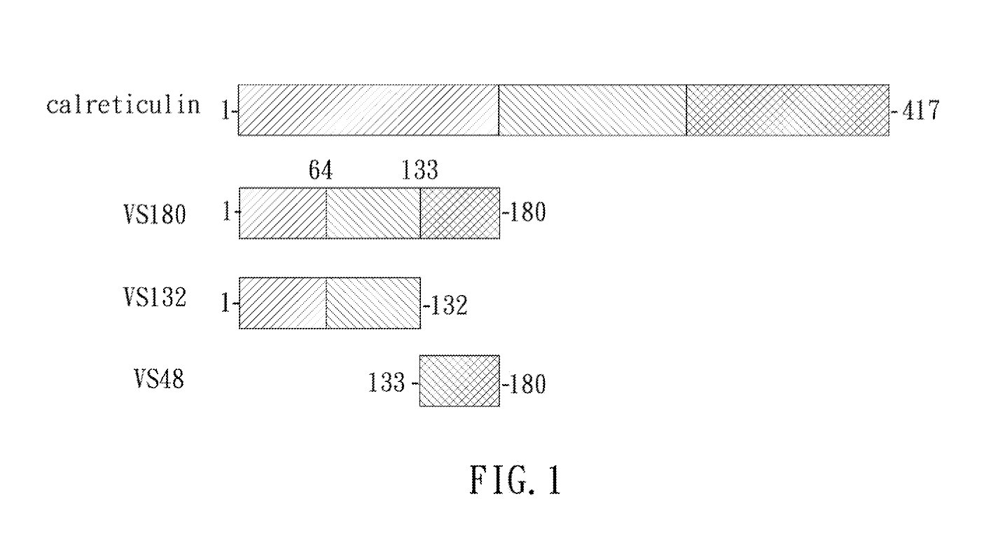

With reference to FIG. 1, a medication for treating choroidal neovascularization according to a preferable embodiment in the present invention is provided, and which comprises an amino acid fragment that derives from N-terminal amino acid sequence of calreticulin (comprising 417 base pairs and 180 amino acids), the 133^{rd} to 180^{th} amino acid sequence from the N-terminal (named as VS48 in the present embodiment) in particular. The sequence of VS48 is set forth in SEQ ID NO: 1, comprising 48 amino acids. In specifically, the VS48 is easy to be delivered to cell, for specifically binding with vascular endothelial cell of eyes, so that the VS48 is sufficient to be applied to vascular endothelial cell of eyes, for inhibiting angiogenesis in eyes, such as migration and tubular formation of vascular endothelial cells, and angiogenesis.

In view of TABLE 1, the solubility of VS48 in water is significantly high, especially in comparison with other fragments that derive from amino acid sequences of calreticulin, VS180 (comprising 1^{st} to 180^{th} amino acid sequence of calreticulin) and VS132 (comprising 1^{st} to 132^{th} amino acid sequence of calreticulin) for example. Accordingly, the VS48 is apt to be dissolved in a solvent directly without any assistance of solubilizers (such as a tag protein, TRX) and with no sediments being produced. The solvent can be any solutions comprising similar osmotic pressure to body's liquid of organisms (such as plasma), for example 0.85-0.9% saline. Preferably, the solvent is 0.85-0.90% saline, which is dramatically stable as store and is capable of being stored for one year under 4° C.

Preferably, the medication for treating choroidal neovascularization according to the preferable embodiment further comprises the said solvent, more preferably for 0.85-0.90% saline, with the VS48 is prepared in the said 0.85-0.90% saline in a concentration of 0.1-1000 μg/ml. In such, the medication for treating choroidal neovascularization of the present embodiment can be manufactured into a type of a drop, being easy and convenient to use by general users.

TABLE 1
Solubility of VS48 and VS 180

| VS fragments | Solubility | |
|---|---|---|
| VS48 | 1000 | μg/ml |
| TRX-VS180 | 500 | μg/ml |
| VS180 | 50 | μg/ml |

Additionally, the medication for treating choroidal neovascularization according to the preferable embodiment further comprises an antiseptic, preferably in a concentration of 1%. The antiseptic is able to prolong storage time of the medication for treating choroidal neovascularization. As an example, the antiseptic can be phenoxetol, which is liquid and is easy to be manufactured into a drop. Also, phenoxetol is safe, especially at 1%, generally leading to no stimulation or immune response to eyes, skin and mucosa. Then, phenoxetol is quite stable under the presence of protein, and will not vapor at room temperature.

In the present embodiment, the VS48 in the medication will specifically bind with vascular endothelial cell, inhibiting migration and tubular formation of vascular endothelial cells, so as to effectively inhibit neovascularization. The VS48 of the present embodiment can be synthesized recombinantly in an appropriate eukaryote system, preferably to combine with a tagged protein showing no immune activity, such as His tagged protein. Otherwise, the tagged protein combined to the VS48 can be further removed from obtained recombinant protein through a preferable removing procedure, following by manufacturing the obtained recombinant protein into medication. With such performance, negative immune responses caused by tagged protein can be completely avoided. Also it is sufficient to make the obtained recombinant protein stably and completely being dissolved in water.

For proving the medication for treating choroidal neovascularization according to the preferable embodiment does have multi-efficacies, including preferable binding ability to organism, reducing negative immune responses caused by recombinant VS48 and convenient in use for user, a serial of trials are carried out. The serial of trials comprise: (A) preparation of VS48; (B) immune response of VS 48 and TRX-VS 48; (C) inhibition of choroidal neovacularization; (D) inhibition of angiogenesis on surface of eyes; (E) binding ability; (F) inhibition of migration and tubular formation; (G) inhibition of angiogenesis in rats.

Trial of (A)

In the present trial, a Raji lymphoma cell line (BCRC 60116) purchased from Bioresource Collection and Research Center in Food Industry Research and Development Institute in Taiwan is prepared to extract total ribonucleic acid therein, followed by amplifying complementary deoxyribonucleic acids (cDNAs) of the vasostatin via reverse transcription polymerase chain reaction, to obtain a DNA fragment of VS48. In the reverse transcription polymerase chain reaction, a primer pair of VS 48 is designed and prepared according to calreticulin cDNAs of endoplasmic reticulum protein. The primer pair of VS 48 comprises deoxyribonucleic acids as set forth in SEQ ID NO: 2.

TABLE 2
The Primer Pair of VS48

| Primer Pair of | |
|---|---|
| VS48 | Sequence |
| Forward Primer | 5′-GCGCATATGCTGCTATCCGTGCCGTTG-3′ |
| Reverse Primer | 5′-GGGCTCGAGCTAGTTGTCTGGCCGCACAA |
| | TCAGTGTGTAC-3′ |

Next, the DNA fragment of VS48 is inserted into an expressed vector (pET32a or pET28a for example, the DNA fragment of VS48 of the present trial is inserted into pET28a comprising His tagged protein), followed by delivering obtained vector (pET32a-VS48 or pET28a-VS48) to E. coli BL21 cells for expressing a recombinant VS48. Then, the recombinant VS48 is purified by nickel-NTA column, processed with protease inhibitor (comprising 1 mM PMSF, 1 μg/ml aprotinin, 1 μg/ml prpstatin and 1 μg/ml leupeptin) and sonicator, followed by centrifuging at 9000 rpm for 20 minutes to take suspension.

The suspension is further purified via affinity chromatography by mixing up the suspension with 10 ml 50% Ni-NTA agarose beads (QIAGEN) that is washed previously with PET solution to obtain a mixture. Then the mixture is kept at 4° C. for 40 minutes and contacted with an affinity column, following by washing the affinity column with 30 ml PET solution. After that, the recombinant VS48 is eluted by 3 ml buffer, and which is further encased in dialysis bag MW 12000-14000 and dialyzed with PBS buffer for 3 hours for condensation. Finally, molecular weight and purity of condensed VS48 are determined by SDS PAGE and coomassie blue stain separately. With such performance, condensed and purified recombinant VS48 is obtained, wherein the recombinant VS48 is combined with His tagged protein.

Trial of (B)

With reference to TABLE 2, for proving that the VS48 will not induce negative immune response to organisms, two groups of experimental animal (with each group comprising eight New Zealand white rabbits being purchased from Taiwan Livestock Research Institute of Council of Agriculture, Executive Yuan) are prepared, including groups B1 and B2. The eight New Zealand white rabbits in the two groups are carried out four injections, with each injection being performed by injecting 0.2 mg/ml recombinant TRX-VS48 and 0.2 mg/ml recombinant His-VS48 respectively every two weeks. After the four injections, blood samples collected from each group is diluted to 1/100, 1/500, 1/1000 and 1/2000, and then is measured under OD₅₇₀.

TABLE 3
Groups Assignment in the Trial (B)

| Groups | Injections |
|---|---|
| B1 | Recombinant TRX-VS48 |
| B2 | Recombinant His-VS48 |

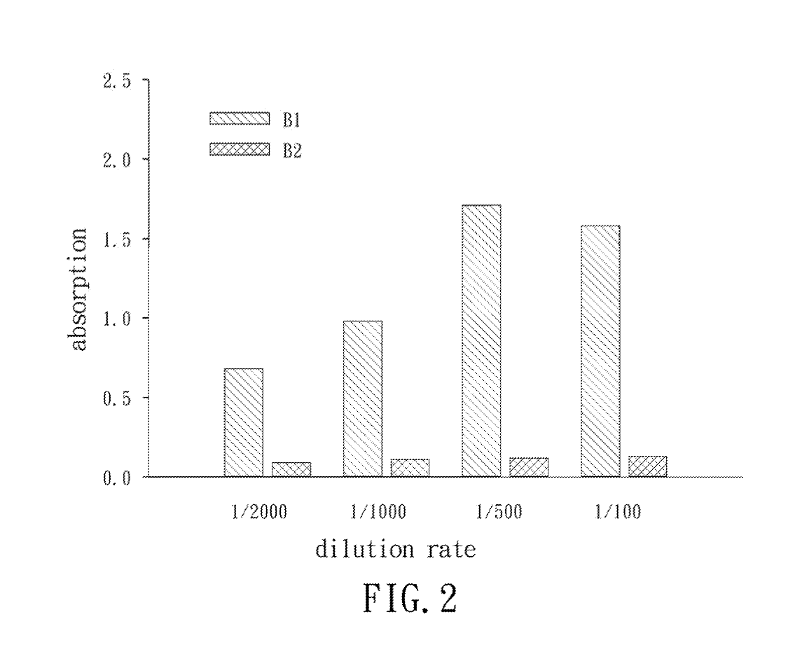

In FIG. 2, it is shown that the recombinant His-VS48 (as being shown in bars of B2) will not lead to immune response to experimental animals, in comparison with the recombinant TRX-VS48 (as being shown in bars of B1).

Trial of (C)

With reference to TABLE 4, for proving that the recombinant VS48 can effectively inhibit choroidal neovascularization, two groups of experimental animal (with each group comprising eight Norway brown rats being purchased from National Laboratory Animal Center in Taiwan) are prepared, including groups C1 and C2. The eight Norway brown rats in the two groups are induced by laser photocoagulation, then given water or 1 μg/ml VS48 formulated with sodium chloride solution respectively to Norway brown rats through eyes since the next day of induction, for twenty days, three times a days (50 μl in volume for each time). After that, eyes of the eight Norway brown rats in the two groups are analyzed via funds fluorescein angiography in the twenty-first, twenty-eighth, thirty-fifth, and forty-second day. Moreover, in the forty-second day, the Norway brown rats in the two groups are dissected to collect eye samples, with samples of each group being identified by hematoxylin-eosin stain, immunofluorescence analysis and von willebrand factor-positive blood vessels.

TABLE 4
Groups Assignment in the Trial (C)

| Groups | Conditions |
|---|---|
| C1 | 0.85% Saline |
| C2 | 1 μg/ml VS48 |

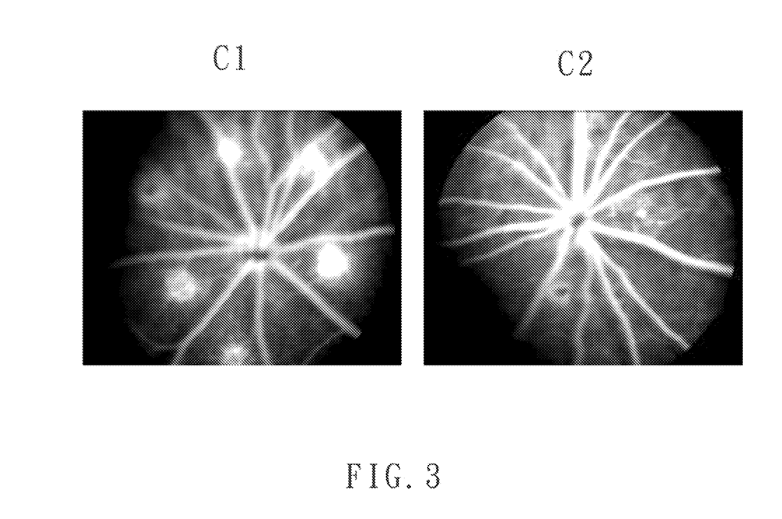

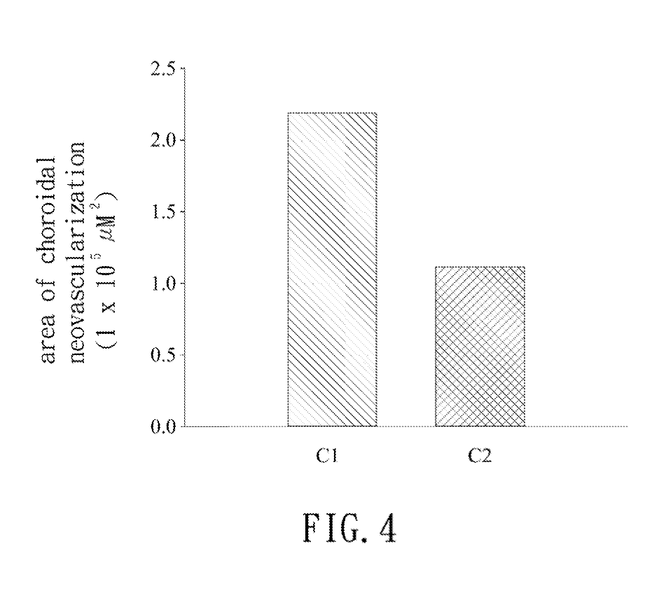

FIGS. 3 and 4 indicate area of choroidal neovascularization observed in the groups C1 and C2. It is apparently that the VS48 is sufficient to reduce choroidal neovascularization in the Norway brown rats (with reference to data of C2), with affected parts of choroidal neovascularization being significant smaller and with tissues in retina being intact in comparison with that of C1.

Trial of (D)

With reference to TABLE 5, for proving that the VS48 is capable of inhibiting angiogenosis occurred on surface of eyes, another two groups of Norway brown rats are prepared and also purchased from National Laboratory Animal Center in Taiwan. The two groups of Norway brown rats include D1 and D2, with each group comprising eight Norway brown rats. In the present trial, the two groups of Norway brown rats are general anesthesia with isoflurane and local anesthesia on eyes with 0.5% proparacaine, followed by disposing hydrogen combined fibroblast growth factor (bFGF) on the eyes of the Norway brown rats, for preciously inducing angiogenosis occurred on surface of eyes. Then, water or 1 μg/ml VS48 formulated with sodium chloride solution are applied to the eyes of the Norway brown rats in the two groups respectively, for fourteen days, three times a days (50 μl in volume for each time). In the fourteenth day, corneal vascular and a distribution there of in the Norway brown rats in the two groups are monitor and reordered.

TABLE 5
Groups Assignment in the Trial (D)

| Groups | Conditions |
|---|---|
| D1 | 0.85% Saline |
| D2 | 1 μg/ml VS48 solution |

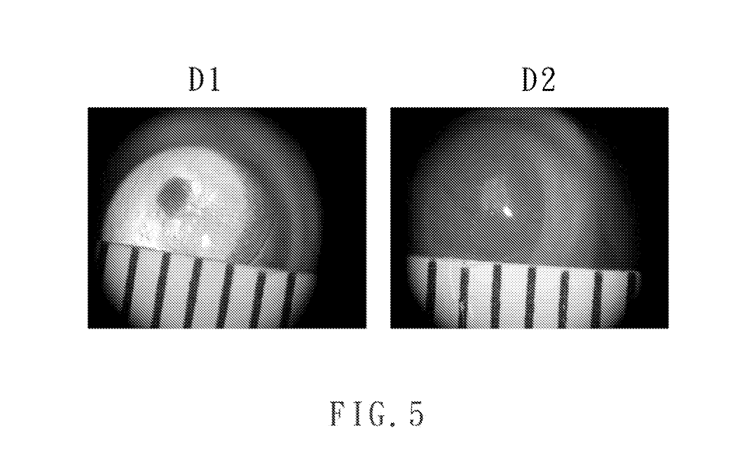

Referring to FIG. 5, it is demonstrated that the VS48 can be applied to affected parts of organism through dropping drops comprising the VS48, dramatically reducing area of corneal hyperplasiareduce induced by angiogenic factors. It is believed that the VS48 shares preferable ability on inhibiting angiogenosis occurred on surface of eyes.

Trial of (E)

With reference to TABLE 6, for proving that the VS48 is capable of specifically binding to vascular endothelial cells to inhibit angiogenosis, a human umbilical vein endothelial cell line (BCRC H-UV001) and a mouse embryonic fibroblast cell line (BCRC 60008) collected from Bioresource Collection and Research Center in Food Industry Research and Development Institute are pre-cultured in a 96-well plate for one day. Next, the human umbilical vein endothelial cell line/mouse embryonic fibroblast cell line is co-cultured with 1-10000 ng/ml VS48 and VS132 respectively, and then analyzed by an anti-His tagged protein antibody. In the present trial, a control groups is also prepared, with the human umbilical vein endothelial cell line/mouse embryonic fibroblast cell line being cultured with general medium, and a binding rate to the cell line in the control group is set up as 100%.

TABLE 6
Groups Assignment in the Trial of (E)

| Groups | Culturing Conditions |
|---|---|
| E1 | 1-10000 ng/ml VS132 |
| E2 | 1-10000 ng/ml VS48 |

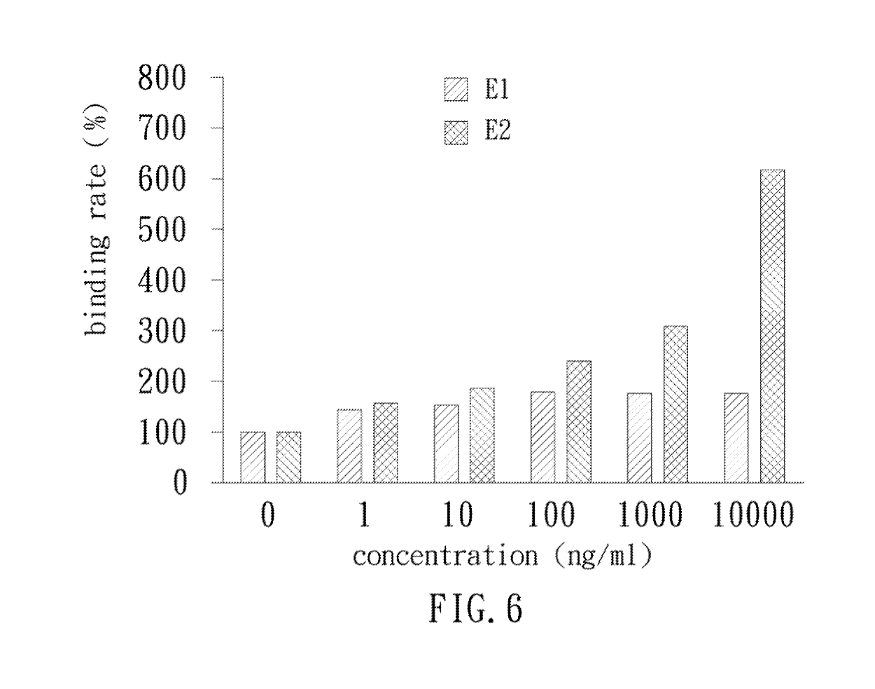

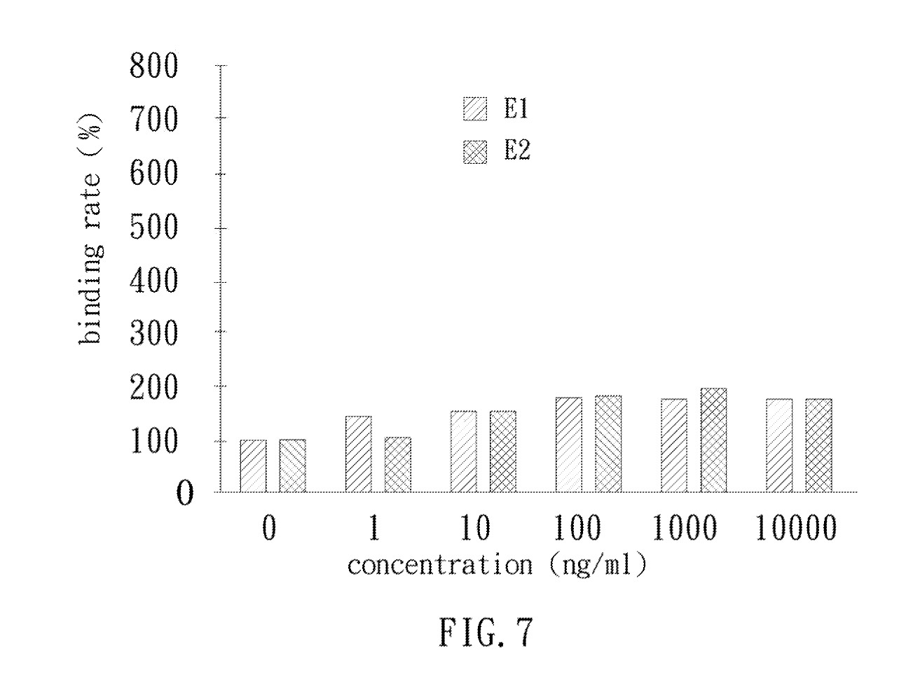

According to FIGS. 6 and 7, the binding rate between the VS48 and the human umbilical vein endothelial cell line is increased by the concentration of the VS48 (with data of the group E2). In comparison, the VS132 does not show specifically binding affinity to the human umbilical vein endothelial cell line and the mouse embryonic fibroblast cell line.

Trial of (F)

For proving that the VS48 achieve inhibitions of angiogenosis through inhibiting migrations and tubular formations in vascular endothelial cell, the present trial is demonstrated.

With reference to TABLE 7, three groups of vascular endothelial cells are prepared in a boyden chamber and co-cultured with 0.85% saline, 100 ng/ml VS132 and 100 ng/ml VS48 respectively, with a sticking factor (for example basic fibroblast growth factor, bFGF) inducting the migration of the vascular endothelial cells. After 24 hours, the vascular endothelial cells of the three groups are further fixed, dyed and then directly examined via microscopy, counting numbers of migrating cells therein.

TABLE 7
Assignment of Groups F1-F3 in the Trial of (F)

| Groups | Conditions |
|---|---|
| F1 | 0.85% saline |
| F2 | 100 ng/ml VS132 |
| F3 | 100 ng/ml VS48 |

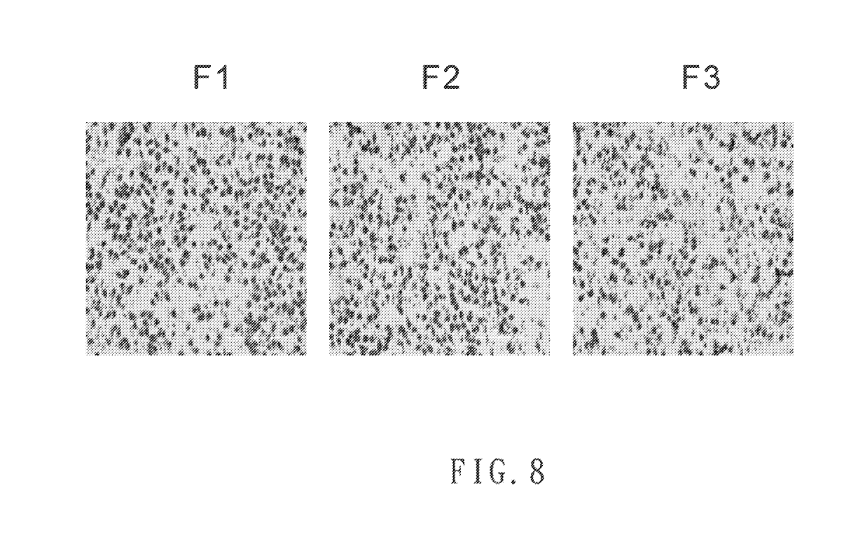

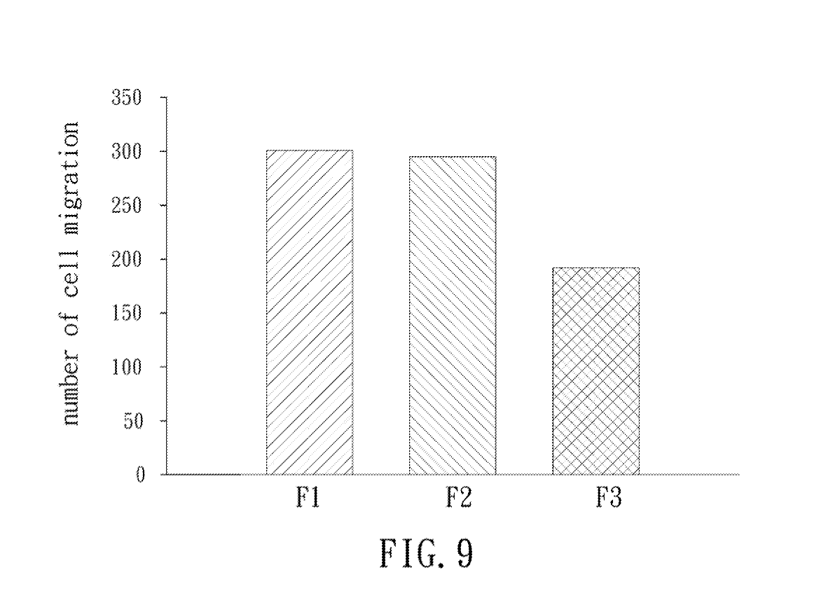

In FIGS. 8 and 9, the VS48 shows great inhibiting ability to vascular endothelial cell migration according to data of groups F1, F2 and F3. Yet, the VS132 is poor in achieving the inhibition of vascular endothelial cell migration.

Next, with reference to TABLE 8, another three groups of vascular endothelial cells are also prepared and co-cultured with 0.85% saline, 100 ng/ml VS132 and 100 ng/ml VS48 for 48 hours. After that, the vascular endothelial cells of the three groups are directly examined via microscopy, determining the tubular formation therein.

TABLE 8
Assignment of Groups F4-F6 in the Trial of (F)

| Groups | Conditions |
|---|---|
| F4 | 0.85% saline |
| F5 | 100 ng/ml VS132 |
| F6 | 100 ng/ml VS48 |

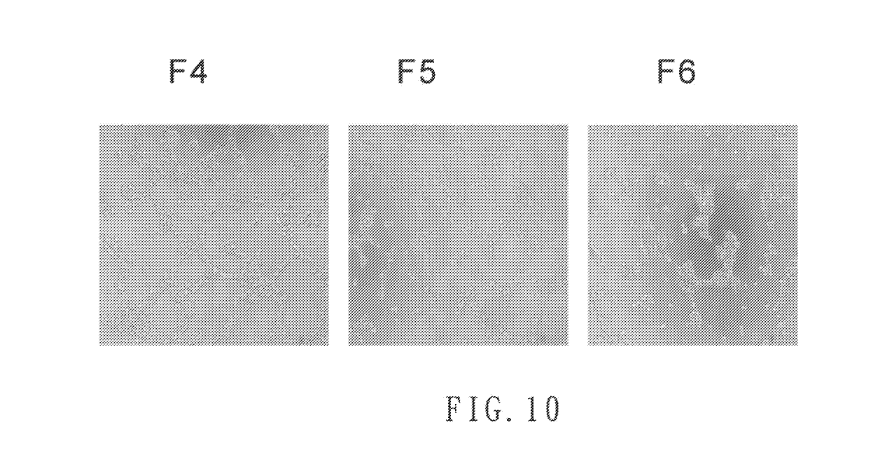

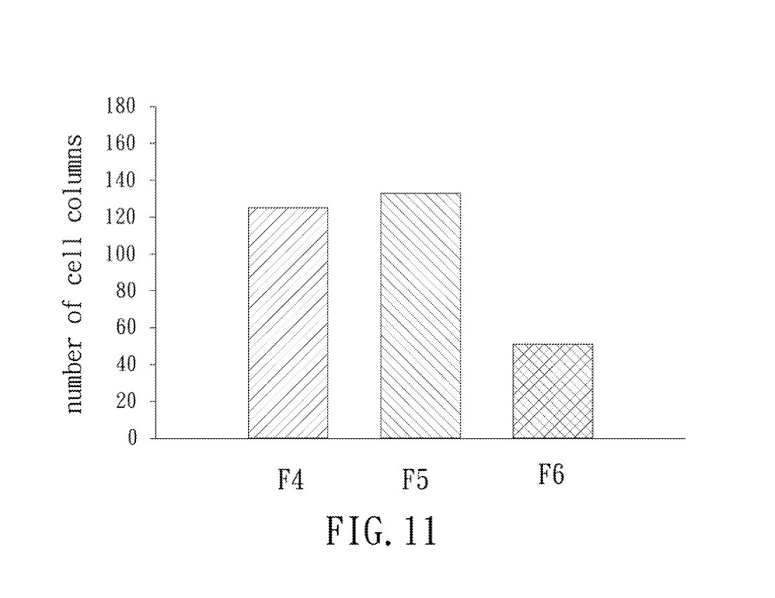

As being shown in FIGS. 10 and 11, it is apparently that the VS48 reduce the tubular formation in the vascular endothelial cells, and in contrast, the VS132 can not achieve that (according to data in groups F4-F6).

Trial of (G)

For proving that the VS48 is capable of inhibiting angiogenosis, an aorta obtained from a Norway brown rat (being purchased from National Laboratory Animal Center in Taiwan) are completely clean, removing connective tissues covered on the aorta, and cut into several pieces (with each piece being 0.2 cm in length). With reference to TABLE 9, the several pieces of the aorta are assigned to three groups (including G1-G3), cultured in a mixture of 1 ml collagen and 3 ml DMEM, and then further cultured with 0.85% saline, 100 ng/ml VS132 and 100 ng/ml VS48 for five days. After that, presences of angiogenosis in each group are monitored and recorded.

TABLE 9
Groups Assignment in the Trial of (G)

| Groups | Conditions |
|---|---|
| G1 | 0.85% saline |
| G2 | 100 ng/ml VS132 |
| G3 | 100 ng/ml VS48 |

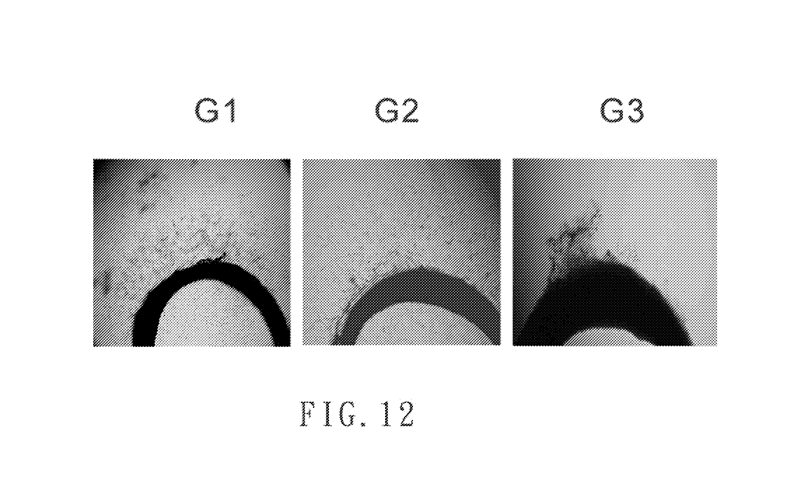

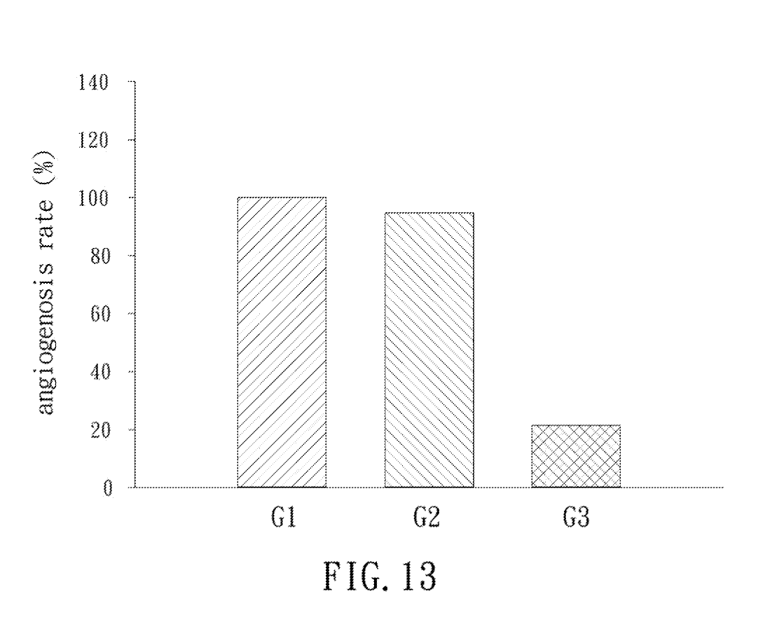

In FIGS. 12 and 13, the aorta of the group G1 is set up as 100% in growing rate. It is noted that the VS48 effectively inhibit the angiogenosis around arterial circle. Yet, the VS132 is poor in achieving the inhibition of angiogenosis.

In summary, the recombinant VS48 can be formulated to 0.1-1000 μg/ml solution and further manufactured into a medication for treating choroidal neovascularization in accordance with the preferable embodiment of the present invention, for inhibiting the angiogenosis either on surface or bottom of eyes. Also, the medication for treating choroidal neovascularization is capable of being manufactured into the type of eye drops, being convenient in practical use even for patients themselves. According to the above trials, it is demonstrated that the VS48 truly can inhibit migrations and tubular formations of the vascular endothelial cell and angiogenosis. Therefore, the medication for treating choroidal neovascularization is capable of reducing angiogenosis, and convenient and easy in use.

Thus since the invention disclosed herein may be embodied in other specific forms without departing from the spirit or general characteristics thereof, some of which forms have been indicated, the embodiments described herein are to be considered in all respects illustrative and not restrictive. The scope of the invention is to be indicated by the appended claims, rather than by the foregoing description, and all changes which come within the meaning and range of equivalency of the claims are intended to be embraced therein.

What is claimed is:

1. A medication for treating choroidal neovascularization comprising:
an amino acid fragment that derives from an angiogenesis inhibiting factor and comprises amino acid sequences as set forth in SEQ ID NO: 1.

2. The medication for treating choroidal neovascularization as claimed in claim 1, further comprising a solvent, with the amino acid fragment being dissolved in the solvent.

3. The medication for treating choroidal neovascularization as claimed in claim 2, wherein the solvent is 0.85-0.90% saline.

4. The medication for treating choroidal neovascularization as claimed in claim 2, wherein 0.1-1000 μg of the amino acid fragment is dissolved in 1 ml of the solvent.

5. The medication for treating choroidal neovascularization as claimed in claim 3, wherein 0.1-1000 μg of the amino acid fragment is dissolved in 1 ml of the solvent.

6. The medication for treating choroidal neovascularization as claimed in claim 1, wherein the medication is in the type of eye drops.

7. A therapy of treating choroidal neovascularization comprising: administering a medication as claimed in claim 1 to an individual in need, with the medication comprising 0.1-1000 μg amino acid fragment having amino acid sequences as set forth in SEQ ID NO: 1 in 1 ml solvent.

8. The therapy of treating choroidal neovascularization as claimed in claim 7, wherein the solvent is 0.85-0.90% saline.
